(19) United States
(12) Patent Application Publication
(10) Pub. No.: US 2011/0002987 A1
(43) Pub. Date: Jan. 6, 2011

(54) Nicorandil Carriers with Enhanced Stability

(75) Inventors: Piero Poli, Lugano (CH); Michela Carcano, Maslianico (IT)

Correspondence Address: Ohlandt, Greeley, Ruggiero & Perle, L.L.P., One Landmark Square, 10th Floor, Stamford, CT, 06901-2682, US

(73) Assignee: RIVOPHARM SA, MANNO (CH)

(21) Appl. No.: 12/920,253

(22) PCT Filed: Mar. 4, 2009
(86) PCT No.: PCT/EP2009/052540

§ 371 Date: Aug. 30, 2010

(30) Foreign Application Priority Data
Mar. 5, 2008 (EP) 08152311.0

(51) Int. Cl. A61K 9/20; A61K 31/4406; A61P 9/08

(52) U.S. Cl. 424/464; 514/355

(57) ABSTRACT

The invention provides a carrier for nicorandil, which is preferably in form of a blister pack, comprising one or several dose blister pockets each containing at least one tablet of nicorandil, and at least one blister pocket containing a molecular sieve.

The present invention relates to nicorandil carrier with enhanced stability.

### BACKGROUND OF THE INVENTION

Nicorandil is a coronary vasodilator endowed with a complex pharmacological activity, being both an organic nitrate and a potassium channel activator. It is used for the therapy of a number of cardiovascular diseases such as myocardial ischemia (especially angina pectoris) and congestive heart failure. The oral drugs containing pharmaceutical compositions of Nicorandil in a solid form suitable for repeated administrations are particularly used in therapy for preventing angina attacks.

As recognized in various patents (EP1001773, U.S. Pat. No. 4,822,808, WO2006/016040), the pharmaceutical compositions of Nicorandil in a solid form are generally characterised by their unsatisfactory stability, especially in the presence of moisture and such other factors as acidity, temperature, light and oxygen.

Particular precautions are therefore required throughout their manufacturing process and during storage, to avoid the products' contact with moisture and to inhibit other degradation processes which may cause an appreciable reduction of the active ingredient content.

Ikorel® tablets, marketed by SANOFI, comprise nicorandil 10 mg or 20 mg, for the prevention and long term treatment of chronic stable angina pectoris.

Ikorel® tablets are presented in soft tempered aluminium foil/PVC blister strips of 10 tablets, in which each tablet is linked to a silica gel capsule desiccant.

The marketing authorisation specifies that the blister should be stored in a dry place below 25° C. Each blister strip should be used within 30 days of opening.

There remains a need for nicorandil formulations or presentations with enhanced stability.

### SUMMARY OF THE INVENTION

The inventors have now shown that replacing the desiccant of the Ikorel® blisters by a molecular sieve surprisingly improved stability of nicorandil.

According to the present invention, there is provided a carrier for nicorandil, containing at least one tablet of nicorandil, and at least a molecular sieve.

In a preferred embodiment, the carrier may be in form of a blister pack, comprising one or several dose blister pockets each containing at least one tablet of nicorandil, and at least one blister pocket containing a molecular sieve.

### DETAILED DESCRIPTION OF THE INVENTION

The Carrier:

The carrier for nicorandil may be any container into which nicorandil tablets can be stored. Preferably, it is a blister pack. However it could also be a plastic or glass bottle or vial, or any suitable container.

The Blister Pack:

Packs in blister pack form for the containment of a unit dose medicaments are envisaged, as are packs containing multiple unit dose blister pockets arranged sequentially or otherwise, such as in series form. A particular multi-unit dose arrangement comprises an elongate strip having multiple blister pockets arranged in series thereon.

The blister pack comprises a base sheet and a lid. The base sheet and lid may comprise the same or different materials.

For ease of manufacturing, and in order to provide the necessary properties to the packaging material, the blisters preferably comprise a nonthermoplastic substrate (such as a metal foil) and a heat sealable layer disposed thereon, and optionally an additional protective layer, such as a polymer film of polyester. The heat sealable layer is usually disposed on the inner surface of the assembled package. The additional protective layer is usually disposed on the surface opposite the heat sealable layer.

The substrate is preferably formed from aluminium foil. However, other metals for the substrate include, but are not limited to, tin, iron, zinc, or magnesium formed on a sheet by vacuum deposition or sputtering and a carboxyl group-containing polymer and/or co-polymer layer formed on the metal layer by lamination.

In one aspect, the blister pack comprises a laminate. Suitably, the laminate comprises material selected from the group consisting of metal foil, organic polymeric material and paper. Suitable metal foils include aluminium or tin foil having a thickness of from 5 to 100 μm, preferably from 10 to 50 μm, such as 20 to 30 μm. Suitable organic polymeric materials include polyethylene, polypropylene, polyvinyl chloride, polychlorotrifluoroethylene, polyethylene terephthalate and combinations thereof.

The heat sealable layer can be formed from any thermoplastic or thermosetting material such as a metal foil, an ionomer resin, polyolefin, or cycloolefin copolymer.

In a preferred embodiment, both the heat sealable layer and the thermoplastic substrate are metal foils, e.g. aluminium foils.

The outer protective layer, if present, can be formed of any material as long as the final laminate has the requisite properties.

Adhesives may be used to join the respective layers of materials together. The adhesive layers are typically substantially smaller in thickness relative to the thickness of the substrate, heat sealable and/or protective layers which they bond.

In a preferred embodiment, the carrier in form of a blister pack comprises 10 or less dose blister pockets each containing one tablet of nicorandil, and one blister pocket containing a molecular sieve. In a preferred embodiment, the blister pocket comprising the molecular sieve is located within a distance inferior to about 10 cm, preferably inferior to about 8 cm, from the blister pockets which contain nicorandil.

It is to be understood that the carrier, be it a blister pack or any other suitable container, does not comprise any silica gel.

The Molecular Sieve:

With the appearance of small opaque pinkish beads, molecular sieves are generally synthetically produced. The molecular sieve material used in the present invention is preferably a metal-alumino silicates or a synthetic polymer gel. Preferred materials include hydroxyapatite, faujasite, calcium silicate, zirconia, zeolite, or the like. Exemplary synthetic polymers include, but are not limited to, stylene-divinylbenzene copolymer, cross-linked polyvinyl alcohol, cross-linked polyacrylate, cross-linked vinyl ether-maleic anhydride copolymer, cross-linked stylene-maleic anhydride copolymer or cross-linked polyamide, and combinations thereof.

In a preferred embodiment, the molecular sieve consists in sodium aluminosilicate.

Molecular sieves have many internal cavities that are linked by window openings of precise diameters. These diameters (measured in Ångstroms) classify molecular sieves—3 Å, 4 Å, 5 Å, and 10 Å (also known as 13X).

Molecular sieves differ from conventional desiccants in the size of these pore openings. While conventional desiccants have a variety of pore size openings, the pore size opening of molecular sieves are all the same size—a “sieve” on the molecular scale. This type of structure enables molecular sieves to screen or select the components which will be adsorbed; for example, adsorption of water while excluding adsorption of valuable organics which might be part of a product's make-up (e.g. perfumes, plasticizers, solvents, etc.)

Adsorption occurs only of molecules with smaller diameters than these cavity openings. Larger molecules are excluded from adsorption. Preferentially adsorbed are molecules of greater polarity.

Molecular sieves adsorb water molecules and other contaminants from liquids and gases down to very low levels—often just 1 part per million.

FIG. 1 is a chart that is useful as a guide for the selection of molecular sieves.

A Molecular Sieve from 3 Å to 8 Å, preferably 4 Å, is preferred in the present invention.

Preferably the molecular sieve is included into a container, as described for instance in EP 824 480. In a preferred embodiment, the container includes a container body that forms at least a partial enclosure so that an inside space and an outside space is created with respect to the container body. There is an insert formed from desiccant entrained thermoplastic that is fixed relative to the container body. At least a portion of the insert is exposed to the inside space of the container body so that it can absorb moisture therefrom. The desiccant entrained thermoplastic from which the insert is constructed has a high desiccant concentration of at least forty percent desiccant to thermoplastic by weight. The container body is constructed from substantially desiccant-free thermoplastic in one embodiment and from low desiccant concentrate thermoplastic having at most twenty percent desiccant to thermoplastic by weight in another embodiment. In a preferred embodiment, the container is constructed from polypropylene.

In a particular embodiment, the molecular sieve may be a Molecular Sieve 4A as provided by CSP technologies, which is made of sodium aluminosilicate, also called synthetic zeolite, in powder form. The formula of the zeolite is Na₂*Al₂O₃*2SiO₂*zH₂O.

Molecular Sieve 4A is protected in a polypropylene resin with an overall width of about 8.2 mm, an overall length of about 17.7 mm, an overall thickness of about 2.5 mm, and shows an absorption capacity of at least 0.0615 g at 22° C. 80% relative humidity, a capacity over 24 hours of at least 0.06 g at 22° C. 80% relative humidity, and a saturation time of 150 to 300 hours at 22° C. 80% relative humidity.

The Figures and examples illustrate the invention without limiting its scope.

### LEGENDS TO THE FIGURES

FIG. 1 is a chart that is useful as a guide for the selection of molecular sieves.

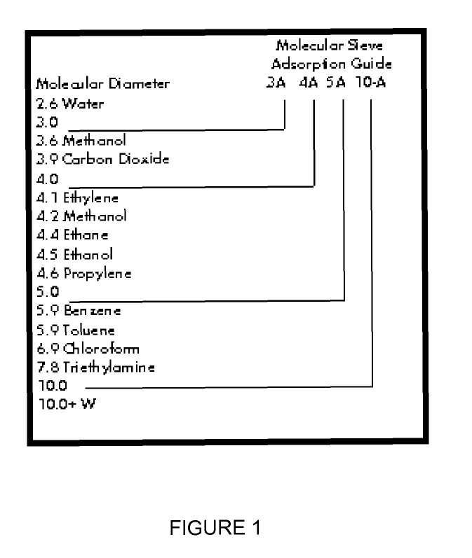

FIGS. 2A and 2B are graphs showing the percentage of impurities (rrt 0.14 for FIG. 2A, rrt 0.25 for FIG. 2B), at 25° C., 60% RH for batch 043/07.

FIGS. 3A and 3B are graphs showing the percentage of impurities (rrt 0.14 for FIG. 3A, rrt 0.25 for FIG. 3B), at 25° C., 60% RH for batch 044/07.

FIGS. 4A and 4B are graphs showing the percentage of impurities (rrt 0.14 for FIG. 4A, rrt 0.25 for FIG. 4B), at 30° C., 65% RH for batch 043/07.

FIGS. 5A and 5B are graphs showing the percentage of impurities (rrt 0.14 for FIG. 5A, rrt 0.25 for FIG. 5B), at 30° C., 65% RH for batch 044/07.

FIGS. 6A and 6B are graphs showing the percentage of impurities (rrt 0.14 for FIG. 6A, rrt 0.25 for FIG. 6B), at 40° C., 75% RH for batch 043/07.

FIGS. 7A and 7B are graphs showing the percentage of impurities (rrt 0.14 for FIG. 7A, rrt 0.25 for FIG. 7B), at 40° C., 75% RH for batch 044/07.

### EXAMPLES

Stability Tests:

Two batches of Nicorandil tablets, 10 mg (batch 043/07) and 20 mg (batch 044/07) respectively, were prepared and packed according to the invention, in alu/alu blister together with a desiccant system.

Silica gel and molecular sieves were compared as desiccant systems and among molecular sieves, systems with different capacity of water absorption were considered.

043/07A: silica gel system

043/07B: 1 lozenge of desiccant plastic composition (Molecular Sieve 4A as provided by CSP technologies), containing molecular sieve 4 Å, a base polymer of polypropylene and an elastomer (moisture absorbed in mg: about 64)

043/07C: 5 lozenges of desiccant plastic composition (Molecular Sieve 4A as provided by CSP technologies), containing molecular sieve 4 Å, a base polymer of polypropylene and an elastomer (moisture absorbed in mg: about 320)

The blisters were placed on stability at the following conditions for 6 months:

25±2° C./60±5% relative humidity (RH)

30±2° C./65±5% RH

40±2° C./75±5% RH

Nicorandil is particularly sensitive to humidity. Two impurities (named rrt0.14 and rrt0.25) are generated when the product is exposed to moisture and therefore were monitored during stability.

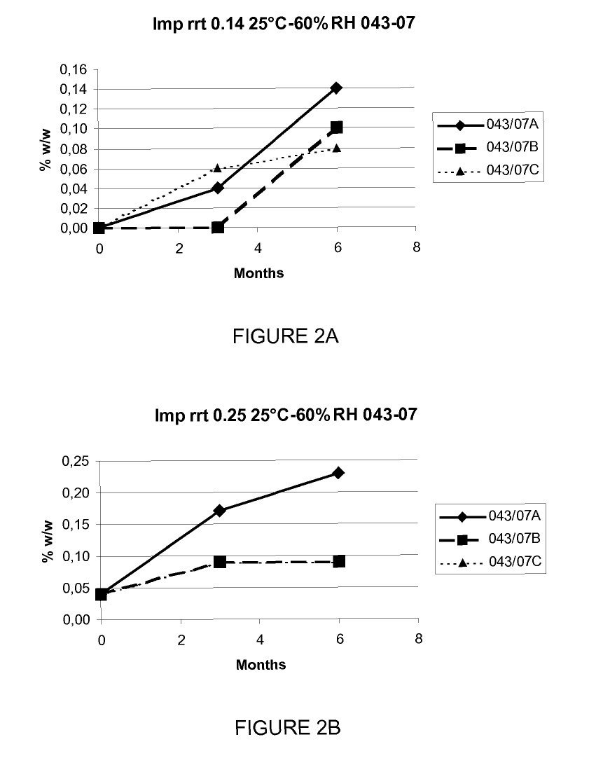

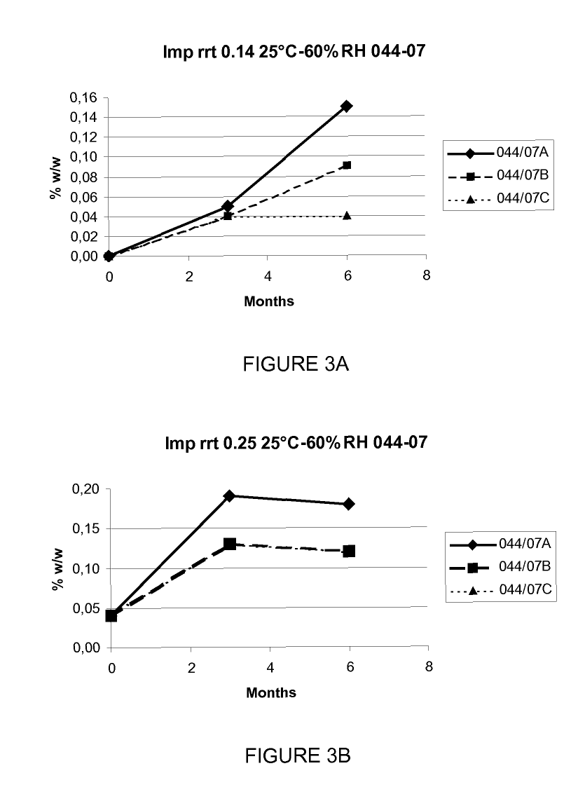

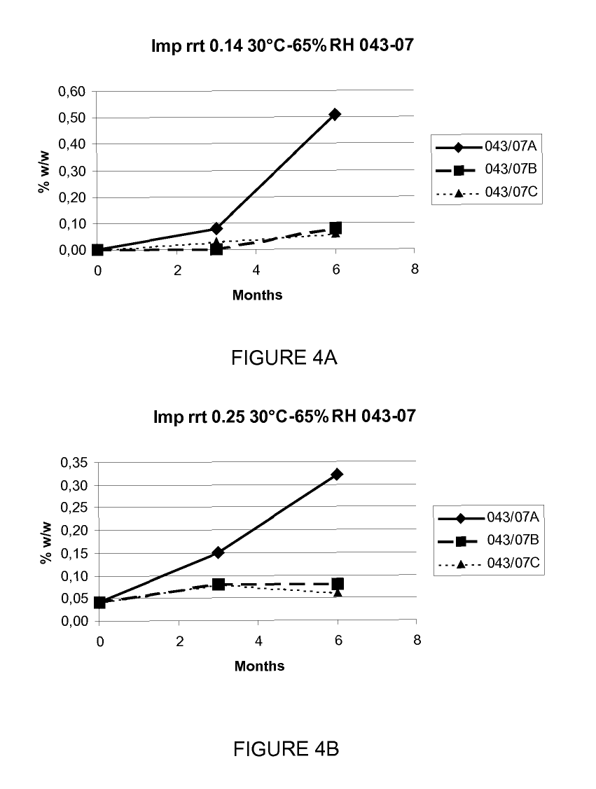

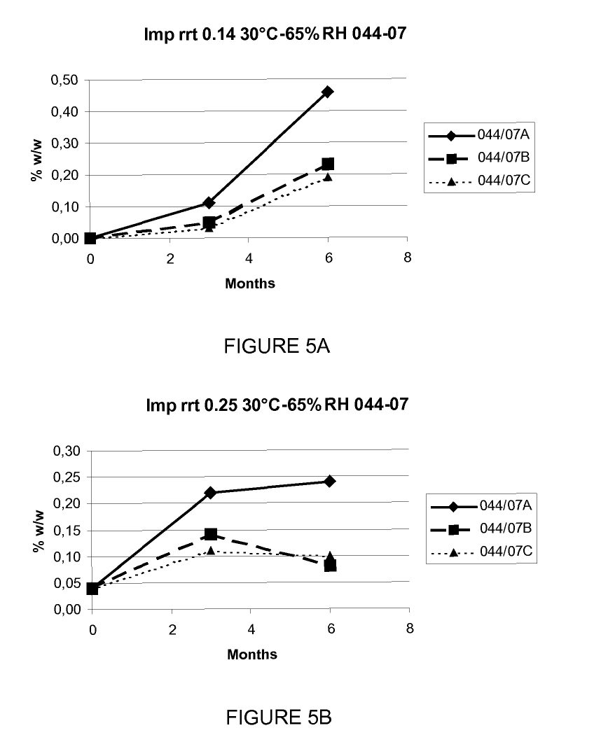

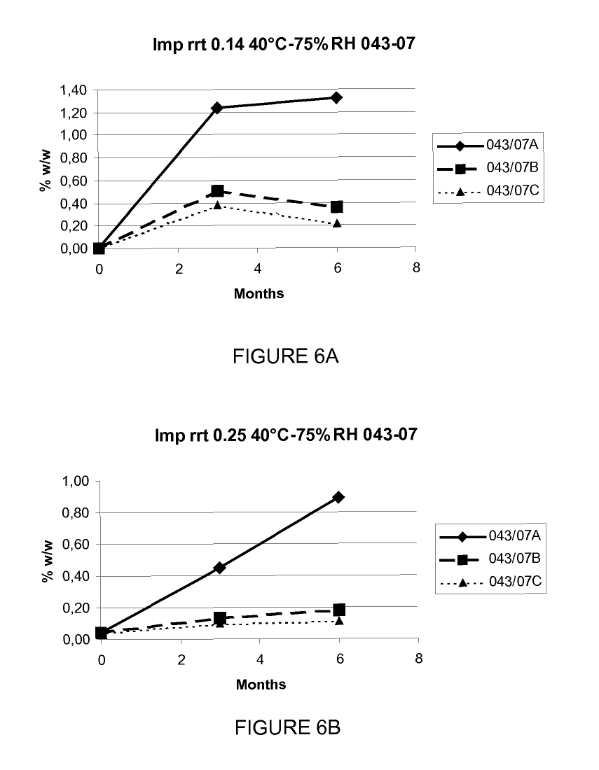

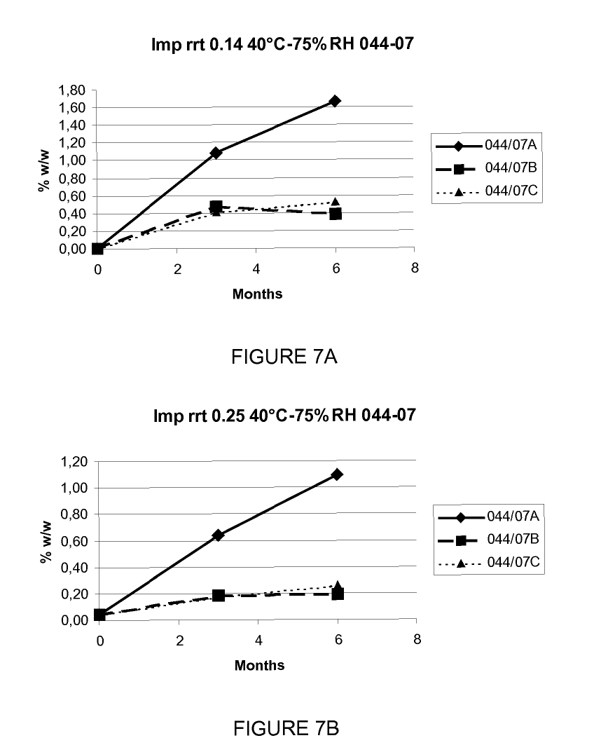

As shown on FIGS. 2 to 7, the level of the impurities is lower when the tablets are stored with the molecular sieves.

Nicorandil is thus more stable when stored in combination with the molecular sieves, as in the blister pack of the invention.

The improved stability was confirmed after twelve months.

1. A carrier for nicorandil, containing at least one tablet of nicorandil, and at least a molecular sieve.

2. The carrier of claim 1, which is in form of a blister pack, comprising one or several dose blister pockets each containing at least one tablet of nicorandil, and at least one blister pocket containing a molecular sieve.

3. The carrier of claim 2, which comprises several dose blister pockets containing nicorandil, and one blister pocket containing a molecular sieve.

4. The carrier of claim 1, wherein the molecular sieve has window openings from 3 Å to 8 Å diameter.

5. The carrier of claim 4, wherein the molecular sieve has window openings of 4 Å diameter.

6. The carrier of claim 1, wherein the molecular sieve consists in sodium aluminosilicate.

7. The carrier of claim 1, wherein the molecular sieve lies within a thermoplastic container.

8. The carrier of claim 7, wherein the thermoplastic container is constructed from polypropylene.

9. The carrier of claim 1, which is a blister pack comprising at least one heat-sealable layer and at least one layer of a metal foil.

10. The carrier of claim 9, wherein at least one layer of a metal foil is a foil of a metal selected from the group consisting of aluminium, tin, iron, zinc and magnesium.

11. The carrier of claim 1, which is in form of a plastic or glass bottle or vial.
